(19) United States
(12) Patent Application Publication
(10) Pub. No.: US 2021/0353366 A1
(43) Pub. Date: Nov. 18, 2021

(54) SYSTEM AND METHOD FOR PLACING SURGICAL INSTRUMENT IN SUBJECT

(71) Applicant: SHANGHAI UNITED IMAGING HEALTHCARE CO., LTD., Shanghai (CN)

(72) Inventors: Hui YIN, Shanghai (CN); Bing TANG, Shanghai (CN)

(73) Assignee: SHANGHAI UNITED IMAGING HEALTHCARE CO., LTD., Shanghai (CN)

(21) Appl. No.: 17/362,911

(22) Filed: Jun. 29, 2021

Related U.S. Application Data
(63) Continuation of application No. PCT/CN2019/129375, filed on Dec. 27, 2019.

(30) Foreign Application Priority Data
Dec. 29, 2018 (CN) 201811638870.6

(51) Int. Cl. A61B 34/20; A61B 90/30; A61B 6/03; A61B 90/13

(52) U.S. Cl. CPC A61B 34/20; A61B 90/30; A61B 2034/2055; A61B 6/037; A61B 90/13; A61B 6/032

(57) ABSTRACT

The present disclosure relates to a system for placing a surgical instrument in a subject may be provided. The system may include: an image obtaining assembly configured to obtain at least one image relating to the subject; a light emitting component configured to emit a light beam towards the subject; and a configuration adjusting component configured to adjust a configuration of the light emitting component such that the light beam matches at least one path in the at least one image. Each of the at least one path indicates at least a portion of planned trajectory information for placing the surgical instrument in the subject in at least one of the at least one image.

### CROSS-REFERENCE TO RELATED APPLICATIONS

This application is a Continuation of International Application No. PCT/CN2019/129375, filed on Dec. 27, 2019, which claims priority to Chinese Patent Application No. 201811638870.6 filed on Dec. 29, 2018, the contents of each of which are hereby incorporated by reference.

### TECHNICAL FIELD

The present disclosure generally relates to a medical system, and more specifically relates to a system and method for placing a surgical instrument (e.g., a nail) in a subject (e.g., a patient) in a medical procedure.

### BACKGROUND

Diseases such as spinal pathologies and disorders result in symptoms including, e.g., deformity, pain, nerve damage, partially or complete loss of mobility, etc. In order to relieve or eliminate the symptoms, current surgical treatment includes decompression and restoration of the abnormal alignment of the spine with concomitant fusion. For illustration purposes, a medical procedure for placing a surgical instrument in the subject is implemented to correct the abnormal alignment of the spine. In some cases, a trajectory (e.g., an entry point, an entry angle, an entry depth, a final position) of the surgical instrument is planned therefor. Then the surgical instrument is placed in the subject according to the trajectory. Usually, the trajectory needs to avoid traversing important tissue (e.g., nerves, blood vessels) for safety.

Currently, a trajectory is planned based on a medical device and a navigation system. In some cases, the medical device includes a medical device with a three-dimensional scanning function (e.g., a CBCT (Cone Beam Computed Tomography)). The process for planning the trajectory based on the medical device and the navigation system is cumbersome. For example, the process includes registering between the position of the medical device and the position of the navigation system, importing images obtained by the medical device into the navigation system, registering such images, adding markers on the surgical instrument, the medical device, and the subject, etc., thereby resulting in low efficiency and poor user experience. Besides, the medical device with a three-dimensional scanning function and/or the navigation system are expensive, resulting in a high cost to acquire such a system. Thus, it may be desirable to provide a method and system for efficient placement or implementation of a surgical instrument (e.g., an implant) in a subject with a relatively low cost.

### SUMMARY

In one aspect of the present disclosure, a system for placing a surgical instrument in a subject may be provided. The system may include: an image obtaining assembly configured to obtain at least one image relating to the subject; a light emitting component configured to emit a light beam towards the subject; and a configuration adjusting component configured to adjust a configuration of the light emitting component such that the light beam matches at least one path in the at least one image, wherein each of the at least one path indicates at least a portion of planned trajectory information for placing the surgical instrument in the subject in at least one of the at least one image.

In some embodiments, the planned trajectory information may include at least one of a planned entry point of the surgical instrument, a planned entry angle of the surgical instrument, a planned entry depth of the surgical instrument, a planned final position of the surgical instrument, a planned range of an entry point of the surgical instrument, a planned range of an entry angle of the surgical instrument, a planned range of an entry depth of the surgical instrument, or a planned range of a final position of the surgical instrument.

In some embodiments, the system may further include an image processing component, and to adjust a configuration of the light emitting component such that the light beam matches at least one path in the at least one image, the image processing component may be configured to display at least one of the at least one image and representation of the light beam on the at least one of the at least one image; and the configuration adjusting component may be configured to adjust the configuration of the light emitting component such that the representation of the light beam coincides with the at least one path in the at least one of the at least one image.

In some embodiments, to adjust a configuration of the light beam such that the light beam matches at least one path in the at least one image, the configuration adjusting component may be further configured to: adjust the configuration of the light emitting component such that the light beam coincides with at least one of the planned entry point of the surgical instrument, the planned entry angle of the surgical instrument, or the planned final position of the surgical instrument.

In some embodiments, the system may further include a surgical instrument placing component configured to place, based at least in part on the light beam, the surgical instrument in the subject.

In some embodiments, to place, based at least in part on the light beam, the surgical instrument in the subject, the surgical instrument placing component may be further configured to: adjust a configuration of the surgical instrument placing component such that the surgical instrument coincides with at least a portion of the light beam; and place the surgical instrument in the subject.

In some embodiments, to place the surgical instrument, the surgical instrument placing component may be further configured to: place a first end of the surgical instrument on an intersection of the light beam and the subject; and place a second end of the surgical instrument along the at least a portion of the light beam.

In some embodiments, the surgical instrument placing component may be further configured to: place the surgical instrument in the subject according to the planned entry depth.

In some embodiments, the image obtaining assembly may include at least one of: an X-ray machine, a computed tomography (CT), a positron emission tomography (PET), a single photon emission computed tomography (SPECT), or a magnetic resonance (MR).

In some embodiments, the X-ray machine may include a C-arm Xray imaging apparatus.

In some embodiments, the image obtaining assembly may further include a frame, wherein the light emitting component is mounted on the frame.

In some embodiments, a cross section of the light beam may have a shape of T, a shape of a crisscross, or a shape of a dot.

In some embodiments, the configuration adjusting component may include a holder mounted on the image obtaining assembly, and the light emitting component is mounted on the holder.

In some embodiments, the configuration adjusting component may include an automation component, the light emitting component may be mounted on the automation component, and the automation component may be configured to automatically adjust the configuration of the light emitting component.

In some embodiments, the automation component may be mounted on the image obtaining assembly or a component external to the image obtaining assembly.

In some embodiments, the surgical instrument may be mounted inside the surgical instrument placing component.

In some embodiments, the surgical instrument placing component may have a shape of a tube, and a center axis of the surgical instrument placing component may overlap a center axis of the surgical instrument.

In some embodiments, the surgical instrument placing component may include a second automation component configured to automatically adjust the configuration of the surgical instrument placing component.

In some embodiments, the surgical instrument may include at least one of a needle, a nail, a screw, or a drill.

In a second aspect of the present disclosure, a method for placing a surgical instrument in a subject may be provided. The method may include: obtaining at least one image of a subject by an image obtaining assembly; causing a light emitting component to emit a light beam towards the subject; adjusting a configuration of the light emitting component such that the light beam matches at least one path in the at least one image, wherein each of the at least one path indicates at least a portion of planned trajectory information for placing the surgical instrument in the subject in at least one of the at least one image.

In some embodiments, the planned trajectory information may include at least one of a planned entry point of the surgical instrument, a planned entry angle of the surgical instrument, a planned entry depth of the surgical instrument, a planned final position of the surgical instrument, a planned range of an entry point of the surgical instrument, a planned range of an entry angle of the surgical instrument, a planned range of an entry depth of the surgical instrument, or a planned range of a final position of the surgical instrument.

In some embodiments, wherein adjusting a configuration of the light emitting component such that the light beam matches at least one path in the at least one image, the method may further include: displaying at least one of the at least one image and representation of the light beam on the at least one of the at least one image; and adjusting the configuration of the light emitting component such that the representation of the light beam coincides with the at least one path in the at least one of the at least one image.

In some embodiments, wherein adjusting a configuration of the light emitting component such that the light beam matches at least one path in the at least one image, the method may further include: adjusting the configuration of the light emitting component such that the light beam coincides with at least one of the planned entry point of the surgical instrument, the planned entry angle of the surgical instrument, or the planned final position of the surgical instrument.

In some embodiments, the method may further include: placing the surgical instrument in the subject based at least in part on the light beam.

In some embodiments, wherein placing the surgical instrument based at least in part on the light beam, the method may further include: adjusting a configuration of the surgical instrument placing component such that the surgical instrument coincides with at least a portion of the light beam; and placing the surgical instrument in the subject.

In some embodiments, wherein placing the surgical instrument in the subject, the method may further include: placing a first end of the surgical instrument on an intersection of the light beam and the subject; and placing a second end of the surgical instrument along the at least a portion of the light beam.

In some embodiments, wherein placing the surgical instrument in the subject, the method may further include: placing the surgical instrument in the subject according to the planned entry depth.

In some embodiments, the image obtaining assembly may include at least one of: an X-ray machine, a computed tomography (CT), a positron emission tomography (PET), a single photon emission computed tomography (SPECT), or a magnetic resonance (MR).

In some embodiments, the X-ray machine may include a C-arm X-ray imaging apparatus.

In some embodiments, the image obtaining assembly may further include a frame, wherein the light emitting component is mounted on the frame.

In some embodiments, a cross section of the light beam may have a shape of T, a shape of a crisscross, or a shape of a dot.

In some embodiments, the surgical instrument may include at least one of a needle, a nail, a screw, or a drill.

In a third aspect of the present disclosure, a non-transitory computer readable medium may be provided. The non-transitory computer readable medium may include instructions being executed by at least one processor, causing the at least one processor to implement a method. The method may include: obtaining at least one image of a subject by an image obtaining assembly; causing a light emitting component to emit a light beam towards the subject; adjusting a configuration of the light emitting component such that the light beam matches at least one path in the at least one image, wherein each of the at least one path indicates at least a portion of planned trajectory information for placing the surgical instrument in the subject in at least one of the at least one image.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary medical system according to some embodiments of the present disclosure;

FIG. 2 is a schematic diagram illustrating exemplary hardware and/or software components of a computing device according to some embodiments of the present disclosure;

FIG. 3 is a schematic diagram illustrating exemplary hardware and/or software components of a mobile device according to some embodiments of the present disclosure;

FIG. 4 is a section view illustrating an exemplary medical system according to some embodiments of the present disclosure;

FIG. 5 is a top view illustrating an exemplary end of a surgical instrument according to some embodiments of the present disclosure;

FIG. 6 is a block diagram illustrating an exemplary processing device according to some embodiments of the present disclosure; and

FIG. 7 is a flowchart illustrating an exemplary process for placing a surgical instrument in a subject according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “unit,” “module,” and/or “block” used herein are one method to distinguish different components, elements, parts, section or assembly of different level in ascending order. However, the terms may be displaced by another expression if they achieve the same purpose.

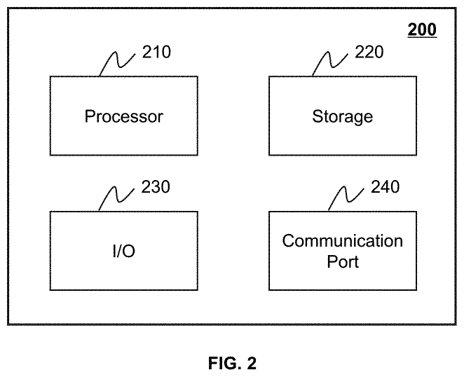

Generally, the word “module,” “unit,” or “block,” as used herein, unless otherwise defined, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices (e.g., processor 210 as illustrated in FIG. 2) may be provided on a computer readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included of connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

A first aspect of the present disclosure relates to a system for placing a surgical instrument (e.g., a nail) in a subject. The system may include an image obtaining assembly, a light emitting component, and a configuration adjusting component. As a preparation for the process of placing the surgical instrument in the subject, the image obtaining assembly may obtain at least one image. The at least one image may be used to perform a trajectory planning to determine a trajectory for placing the surgical instrument in the subject. During the process of placing the surgical instrument in the subject, the configuration adjusting component may be configured to adjust a light beam emitted by the light emitting component such that the light beam coincides with the planned trajectory. The surgical instrument may be placed along the planned trajectory and match at least a portion of the light beam. In some embodiments, the system may also include a surgical instrument placing component configured to place the surgical instrument along the planned trajectory.

In some embodiments of the present disclosure, the light emitting component may be a laser generator (e.g., a crisscross laser generator). Thus the light emitting component may be relatively cheap compared to a current navigation system. Besides, the image obtaining assembly may be a medical device with a two-dimensional scanning function, which is relatively cheaper than a medical device with a three-dimensional scanning function.

A second aspect of the present disclosure relates to a method for placing a surgical instrument in a subject. As a preparation for the process of placing the surgical instrument in the subject, the method may include obtaining at least one image of the subject by an image obtaining assembly. The at least one image may be used to perform a trajectory planning to determine a trajectory for placing the surgical instrument in the subject. During the process of placing the surgical instrument in the subject, the method may include adjusting a light beam emitted by a light emitting component such that the light beam coincides with the planned trajectory. Further, the method may include placing the surgical instrument along the planned trajectory by matching at least a portion of the light beam.

Using the light beam (e.g., a light beam) to indicate the planned trajectory, the surgical instrument may be conveniently placed according to the planned trajectory by matching the surgical instrument with the light beam, thereby improving efficiency and user experience compared to the process for placing the surgical instrument based on a current navigation system.

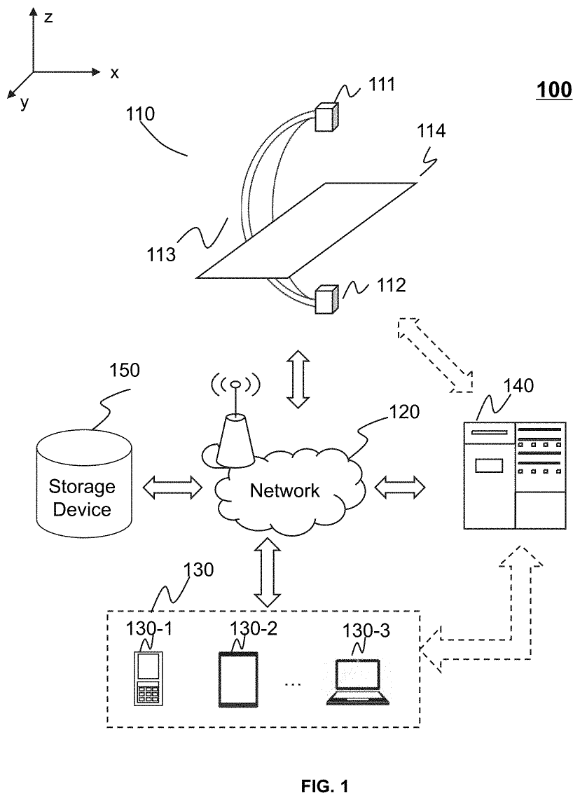

FIG. 1 is a schematic diagram illustrating a medical system 100 according to some embodiments of the present disclosure. The medical system 100 may include an image obtaining assembly 110, a network 120, one or more terminals 130, a processing device 140, and a storage device 150. The components of the medical system 100 may be connected in various ways. Mere by way of example, the image obtaining assembly 110 may be connected to the processing device 140 through the network 120. As another example, the image obtaining assembly 110 may be connected to the processing device 140 directly as indicated by the bi-directional arrow in dotted lines linking the image obtaining assembly 110 and the processing device 140. As a further example, the storage device 150 may be connected to the processing device 140 directly or through the network 120. As still a further example, the terminal 130 may be connected to the processing device 140 directly (as indicated by the bi-directional arrow in dotted lines linking the terminal 130 and the processing device 140) or through the network 120.

The image obtaining assembly 110 may include an X-ray source 111 (also referred to as “X-ray tube”), a detection component 112 (also referred to as “X-ray detector”), an arm 113, and a platform 114. The X-ray source 111 and the detection component 112 may be mounted on the arm 113. The arm 113 may be a C-shaped arm, a G-shaped arm, etc. The X-ray tube 111 and the X-ray detector 112 may be mounted on the arm 113. The arm 113 may include a first end and a second end arranged opposite to each other. For illustration purposes, the X-ray source 111 may be mounted on the first end of the arm 113 and the detection component 112 may be mounted on the second end of the arm 113. For example, the imaging obtaining assembly 110 may include an X-ray machine, e.g., a C-arm X-ray imaging apparatus.

The platform 114 may hold or support a subject. The subject may be a biological subject (e.g., a patient, an animal) or a non-biological subject (e.g., a man-made subject). The X-ray source 111 may emit X-rays (also referred to as “X-ray beam”) towards the subject, and the X-rays may attenuate when passing through the subject. The detection component 112 may receive the attenuated X-rays that pass through the subject and generate readings (also referred to as “imaging data”) corresponding to the received X-rays. In some embodiments, the detection component 112 may include a scintillation detector (e.g., a cesium iodide detector), a gas detector, a circular detector, a square detector, an arcuate detector, or the like, or any combination thereof. The detection component 112 may be a single-row detector or a multiple-row detector.

The network 120 may facilitate the exchange of information and/or data. In some embodiments, one or more components of the medical system 100 (e.g., the image obtaining assembly 110, the terminal 130, the processing device 140, or the storage device 150) may send information and/or data to another component(s) in the medical system 100 via the network 120. For example, the processing device 140 may obtain a user instruction from the terminal 130 via the network 120. As another example, the processing device 140 may send an instruction to the image obtaining assembly 110 via the network 120. The instruction may include an instruction for controlling the X-ray tube 111 to emit X-rays. In some embodiments, the network 120 may be any type of wired or wireless network, or combination thereof. The network 120 may be and/or include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network), a cellular network (e.g., a Long Term Evolution (LTE) network), a frame relay network, a virtual private network (“VPN”), a satellite network, a telephone network, routers, hubs, switches, server computers, and/or any combination thereof. Mere by way of example, the network 120 may include a cable network, a wireline network, an optical fiber network, a telecommunications network, an intranet, an Internet, a local area network (LAN), a wide area network (WAN), a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 120 may include one or more network access points. For example, the network 120 may include wired or wireless network access points such as base stations and/or internet exchange points through which one or more components of the medical system 100 may be connected to the network 120 to exchange data and/or information.

The terminal 130 may include a mobile device 130-1, a tablet computer 130-2, a laptop computer 130-3, or the like, or any combination thereof. In some embodiments, the mobile device 130-1 may include a smart home device, a wearable device, a smart mobile device, a virtual reality device, an augmented reality device, or the like, or any combination thereof. In some embodiments, the smart home device may include a smart lighting device, a control device of an intelligent electrical apparatus, a smart monitoring device, a smart television, a smart video camera, an interphone, or the like, or any combination thereof. In some embodiments, the wearable device may include a bracelet, footgear, eyeglasses, a helmet, a watch, clothing, a backpack, an accessory, or the like, or any combination thereof. In some embodiments, the smart mobile device may include a smartphone, a personal digital assistant (PDA), a gaming device, a navigation device, a point of sale (POS) device, or the like, or any combination thereof. In some embodiments, the virtual reality device and/or the augmented reality device may include a virtual reality helmet, a virtual reality glass, a virtual reality patch, an augmented reality helmet, an augmented reality glass, an augmented reality patch, or the like, or any combination thereof. For example, the virtual reality device and/or the augmented reality device may include a Google Glass, an Oculus Rift, a HoloLens, a Gear VR, etc. In some embodiments, the terminal 130 may remotely operate the image obtaining assembly 110. In some embodiments, the terminal 130 may operate the image obtaining assembly 110 via a wireless connection. In some embodiments, the terminal 130 may receive information and/or instructions inputted by a user, and send the received information and/or instructions to the image obtaining assembly 110 or to the processing device 140 via the network 120. In some embodiments, the terminal 130 may receive data and/or information from the processing device 140. In some embodiments, the terminal 130 may be part of the processing device 140. In some embodiments, the terminal 130 may be omitted.

In some embodiments, the processing device 140 may process data obtained from the image obtaining assembly 110, the terminal 130, or the storage device 150. In some embodiments, the processing device 140 may process data obtained from the image obtaining assembly 110, the terminal 130, or the storage device 150. For example, the processing device 140 may obtain an image related to a subject. The processing device 140 may further determine a path in the image. As used herein, a path in an image of a subject may represent at least a portion of a planned trajectory for placing a surgical instrument in the subject. The processing device 140 may be a central processing unit (CPU), a digital signal processor (DSP), a system on a chip (SoC), a microcontroller unit (MCU), or the like, or any combination thereof. In some embodiments, the processing device 140 may be a single server or a server group. The server group may be centralized or distributed. In some embodiments, the processing device 140 may be local or remote. For example, the processing device 140 may access information and/or data stored in the image obtaining assembly 110, the terminal 130, and/or the storage device 150 via the network 120. As another example, the processing device 140 may be directly connected to the image obtaining assembly 110 (as illustrated by the dashed bidirectional arrow linking the image obtaining assembly 110 and the processing device 140 in FIG. 1), the terminal 130 (as illustrated by the dashed bidirectional arrow linking the terminal 130 and the processing device 140 in FIG. 1), and/or the storage device 150, to access information and/or data. In some embodiments, the processing device 140 may be implemented on a cloud platform. Mere by way of example, the cloud platform may include a private cloud, a public cloud, a hybrid cloud, a community cloud, a distributed cloud, an inter-cloud, a multi-cloud, or the like, or any combination thereof. In some embodiments, the processing device 140 may be implemented on a computing device 200 having one or more components illustrated in FIG. 2 in the present disclosure.

The storage device 150 may store data and/or instructions. In some embodiments, the storage device 150 may store data obtained from the terminal 130 and/or the processing device 140. In some embodiments, the storage device 150 may store data and/or instructions that the processing device 140 may execute or use to perform exemplary methods described in the present disclosure. In some embodiments, the storage device 150 may include a mass storage, removable storage, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memory may include a random-access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (PEROM), an electrically erasable programmable ROM (EEPROM), a compact disk ROM (CD-ROM), and a digital versatile disk ROM, etc. In some embodiments, the storage device 150 may be implemented on a cloud platform. Mere by way of example, the cloud platform may include a private cloud, a public cloud, a hybrid cloud, a community cloud, a distributed cloud, an inter-cloud, a multi-cloud, or the like, or any combination thereof.

In some embodiments, the storage device 150 may be connected to the network 120 to communicate with one or more components of the medical system 100 (e.g., the terminal 130, the processing device 140). One or more components of the medical system 100 may access the data or instructions stored in the storage device 150 via the network 120. In some embodiments, the storage device 150 may be directly connected to or communicate with one or more components of the medical system 100 (e.g., the terminal 130, the processing device 140). In some embodiments, the storage device 150 may be part of the processing device 140.

It should be noted that the above description of the image obtaining assembly 110 is provided for the purposes of illustration, and is not intended to limit the scope of the present disclosure. For persons having ordinary skills in the art, multiple variations and modifications may be made under the teachings of the present disclosure. However, those variations and modifications shall not depart from the scope of the present disclosure. For example, the image obtaining assembly 110 may be another kind of medical imaging device, including a positron emission tomography (PET) device, a single photon emission computed tomography (SPECT) device, a computed tomography (CT) device (e.g., a Cone Beam CT (CBCT), a Magnetic Resonance Imaging (MRI) device, a PET-CT device, a PET-MRI device, or a SPECT-MRI device. Merely by way of example, the X-ray tube 111 and the X-ray detector 112 of the image obtaining assembly 110 may be replaced by a transmitting coil and a receiving coil, and the image obtaining assembly 110 may be employed in an MRI device.

FIG. 2 is a schematic diagram illustrating exemplary hardware and/or software components of a computing device 200 on which the processing device 140 may be implemented according to some embodiments of the present disclosure. As illustrated in FIG. 2, the computing device 200 may include a processor 210, a storage 220, an input/output (I/O) 230, and a communication port 240.

The processor 210 may execute computer instructions (program code) and, when executing the instructions, cause the processing device 140 to perform functions in accordance with techniques described herein. The computer instructions may include, for example, routines, programs, objects, components, signals, data structures, procedures, modules, and functions, which perform particular functions described herein. In some embodiments, the processor 210 may process data and/or images obtained from the image obtaining assembly 110, the terminal 130, the storage device 150, and/or any other component of the medical system 100. The processor 210 may process data obtained from the image obtaining assembly 110, the terminal 130, or the storage device 150. For example, the processing device 140 may obtain an image related to a subject. The processing device 140 may further determine a path in the image. As used herein, a path in an image of a subject may represent at least a portion of a planned trajectory for placing a surgical instrument in the subject. In some embodiments, the processor 210 may include one or more hardware processors, such as a microcontroller, a microprocessor, a reduced instruction set computer (RISC), an application specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuit or processor capable of executing one or more functions, or the like, or any combinations thereof.

Merely for illustration, only one processor is described in the computing device 200. However, it should be noted that the computing device 200 in the present disclosure may also include multiple processors. Thus operations and/or method steps that are performed by one processor as described in the present disclosure may also be jointly or separately performed by the multiple processors. For example, if in the present disclosure the processor of the computing device 200 executes both process A and process B, it should be understood that process A and process B may also be performed by two or more different processors jointly or separately in the computing device 200 (e.g., a first processor executes process A and a second processor executes process B, or the first and second processors jointly execute processes A and B).

The storage 220 may store data/information obtained from the image obtaining assembly 110, the terminal 130, the storage device 150, or any other component of the medical system 100. In some embodiments, the storage 220 may include a mass storage device, removable storage device, a volatile read-and-write memory, a read-only memory (ROM), or the like, or any combination thereof. For example, the mass storage may include a magnetic disk, an optical disk, a solid-state drive, etc. The removable storage may include a flash drive, a floppy disk, an optical disk, a memory card, a zip disk, a magnetic tape, etc. The volatile read-and-write memory may include a random access memory (RAM). The RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. The ROM may include a mask ROM (MROM), a programmable ROM (PROM), an erasable programmable ROM (PEROM), an electrically erasable programmable ROM (EEPROM), a compact disk ROM (CD-ROM), and a digital versatile disk ROM, etc. In some embodiments, the storage 220 may store one or more programs and/or instructions to perform exemplary methods described in the present disclosure.

The I/O 230 may input or output signals, data, and/or information. In some embodiments, the I/O 230 may enable a user interaction with the processing device 140. In some embodiments, the I/O 230 may include an input device and an output device. Exemplary input devices may include a keyboard, a mouse, a touch screen, a microphone, or the like, or a combination thereof. Exemplary output devices may include a display device, a loudspeaker, a printer, a projector, or the like, or a combination thereof. Exemplary display devices may include a liquid crystal display (LCD), a light-emitting diode (LED)-based display, a flat panel display, a curved screen, a television device, a cathode ray tube (CRT), or the like, or a combination thereof.

The communication port 240 may be connected to a network (e.g., the network 120) to facilitate data communications. The communication port 240 may establish connections between the processing device 140 and the image obtaining assembly 110, the terminal 130, or the storage device 150. The connection may be a wired connection, a wireless connection, or a combination of both that enables data transmission and reception. The wired connection may include an electrical cable, an optical cable, a telephone wire, or the like, or any combination thereof. The wireless connection may include Bluetooth, Wi-Fi, WiMAX, WLAN, ZigBee, mobile network (e.g., 3G, 4G, 5G, etc.), or the like, or a combination thereof. In some embodiments, the communication port 240 may be a standardized communication port, such as RS232, RS485, etc. In some embodiments, the communication port 240 may be a specially designed communication port. For example, the communication port 240 may be designed in accordance with the digital imaging and communications in medicine (DICOM) protocol.

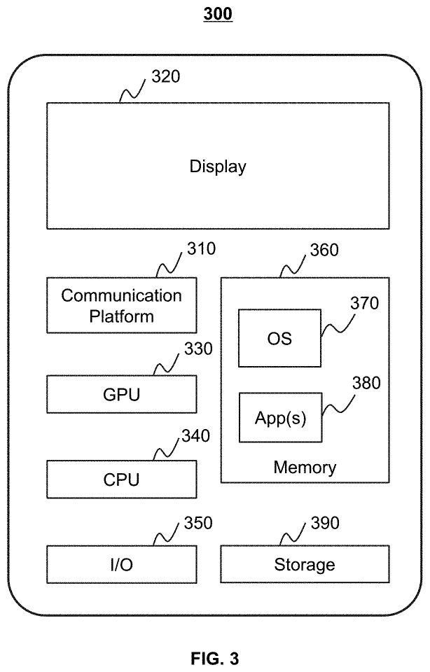

FIG. 3 is a schematic diagram illustrating exemplary hardware and/or software components of a mobile device 300 according to some embodiments of the present disclosure. As illustrated in FIG. 3, the mobile device 300 may include a communication platform 310, a display 320, a graphics processing unit (GPU) 330, a central processing unit (CPU) 340, an I/O 350, a memory 360, and a storage 390. In some embodiments, any other suitable component, including but not limited to a system bus or a controller (not shown), may also be included in the mobile device 300. In some embodiments, a mobile operating system 370 (e.g., iOS, Android, Windows Phone, etc.) and one or more applications 380 may be loaded into the memory 360 from the storage 390 in order to be executed by the CPU 340. The applications 380 may include a browser or any other suitable mobile apps for receiving and rendering information relating to image processing or other information from the processing device 140. User interactions with the information stream may be achieved via the I/O 350 and provided to the processing device 140 and/or other components of the medical system 100 via the network 120.

To implement various modules, units, and their functionalities described in the present disclosure, computer hardware platforms may be used as the hardware platform(s) for one or more of the elements described herein. The hardware elements, operating systems and programming languages of such computers are conventional in nature, and it is presumed that those skilled in the art are adequately familiar therewith to adapt those technologies to place a surgical instrument in a subject as described herein. A computer with user interface elements may be used to implement a personal computer (PC) or another type of work station or terminal device, although a computer may also act as a server if appropriately programmed. It is believed that those skilled in the art are familiar with the structure, programming and general operation of such computer equipment and as a result, the drawings should be self-explanatory.

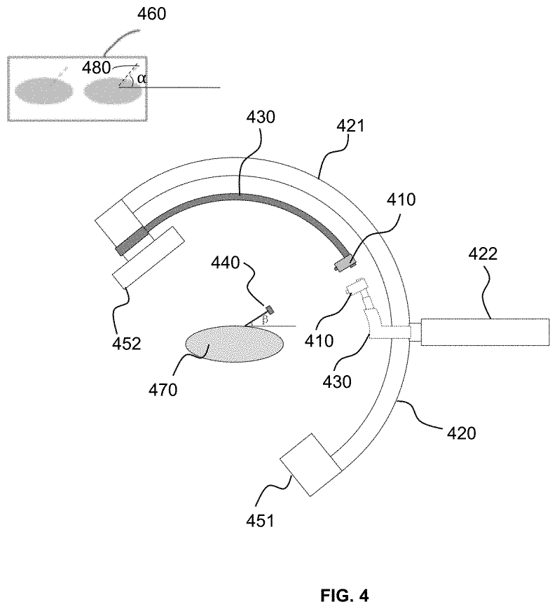

FIG. 4 is a section view illustrating an exemplary medical system 100 according to some embodiments of the present disclosure. The medical system 100 may include a light emitting component 410, an image obtaining assembly 420, and a surgical instrument placing component 440.

The image obtaining assembly 420 may be the same as or similar to the image obtaining assembly 110. As illustrated in FIG. 4, the image obtaining assembly 420 may include an X-ray source 451 (also referred to as “X-ray tube”), a detection component 452, and a frame. The X-ray source 451 and the detection component 452 may be mounted on the frame. The frame may include a first arm 421 and a second arm 422. The first arm 421 may be able to rotate around a center thereof. The first arm 421 may be a C-shaped arm. The second arm 422 may be connected to the first arm 421. The second arm 422 may be moveable along an axis (e.g., a center axis along a horizontal direction) thereof. In some embodiments, the second arm 422 may have at least one slide rail. The first arm 421 may slide through the at least one slide rail.

The image obtaining assembly 420 may also include a platform (not shown in FIG. 4). The platform may hold or support a subject 470. The subject 470 may change his or her posture based on practical demands. For example, the subject 470 may lie on the back on the platform. As another example, the subject 470 may lie on the side on the platform. The X-ray source 451 may emit X-rays (also referred to as “X-ray beam”) towards the subject 470, and the X-rays may attenuate when passing through the subject 470. The detection component 452 may receive the attenuated X-rays that pass through the subject 470 and generate readings (also referred to as “imaging data”) corresponding to the received X-rays. The image obtaining assembly 420 may obtain an image based on the attenuated X-rays. As used herein, the image may represent an anatomical structure of at least a portion (e.g., an organ, tissue) of the subject 470. The image may be a two-dimensional (2D) image. In some embodiments, if the image obtaining assembly 420 is another kind of medical imaging device (e.g., a CT device), the image may be a three-dimensional (3D) image.

In some embodiments, the image obtaining assembly 420 may obtain images of different views. In some embodiments, the image obtaining assembly 420 may obtain the images of the different views by rotating the first arm 421. For example, the first arm 421 may be rotated to a front of the subject 470 to obtain an image of a front view (or referred to as a front image for brevity) of the subject 470. As used herein, the image obtaining assembly 420 may obtain the front image when the subject 470 lies on the back on the platform (e.g., along the X-direction as illustrated in FIG. 1), the X-ray source 451 and the detection component 452 are situated along the Z direction as illustrated in FIG. 1. As another example, the first arm 421 may be rotated to a side of the subject 470 to obtain an image of a side view (or referred to as a lateral image for brevity) of the subject 470. As used herein, the image obtaining assembly 420 may obtain the lateral image when the subject 470 lies on the side on the platform, the X-ray source 451 and the detection component 452 are situated along the X direction or the Y direction as illustrated in FIG. 1. In some embodiments, the first arm 421 may rotate by 90 degrees to move from the front to the side of the subject 470.

In some embodiments, the subject 470 may change his or her posture to facilitate the acquisition of images of different views. For example, the first arm 421 may stay in the same position, e.g., along the Z direction, and the front image and the lateral image may be obtained when the subject 470 lies on the back on the platform or lies on the side on the platform, respectively.

The image obtaining assembly 420 may assist in implementing a medical procedure for placing a surgical instrument in the subject 470. In some embodiments, the medical procedure may be performed to, e.g., correct an abnormal alignment of at least a portion of the subject 470, ablate at least a portion of the subject 470, etc. For illustration purposes, the surgical instrument may include a needle, a nail, a screw, a drill, etc. The surgical instrument may be made of a biocompatible material. For example, the biocompatible material may include a metallic material, a polymeric material, ceramics, a bone material, or the like, or any combination thereof.

Before the initiation of the medical procedure, the image obtaining assembly 420 may obtain at least one image. Each of the at least one image may be obtained as the process described above. The at least one image may correspond to at least one view. In some embodiments, the at least one image may be obtained from at least one selected view according to practical demands. For example, the at least one image may include the front image obtained from the front view and the lateral image obtained from the side view.

The at least one image may be used to determine a treatment plan for the medical procedure. For illustration purposes, the treatment plan may include a planned trajectory for placing the surgical instrument in the subject 470, planned trajectory information for placing the surgical instrument in the subject 470, a length of the surgical instrument, a configuration of the light emitting component 410, a configuration of a light beam emitted by the light emitting component 410, etc. In some embodiments, the configuration of the light beam may coincide with the configuration of the light emitting component 410. For example, the configuration of the light beam may include an orientation of the light beam, an emitting angle of the light beam, an emitting direction of the light beam, etc.

As used herein, the planned trajectory and/or the planned trajectory information may indicate how the surgical instrument needs to be placed in the subject 470 during the medical procedure. For example, the planned trajectory information may include a planned entry point (e.g., a point in a three-dimensional space) of the surgical instrument, a planned entry angle of the surgical instrument, a planned entry depth of the surgical instrument, a planned final position (e.g., a position in a three-dimensional space) of the surgical instrument (collectively referred to as “first planned trajectory information”), a planned range of an entry point of the surgical instrument, a planned range of an entry angle of the surgical instrument, a planned range of an entry depth of the surgical instrument, a planned range of a final position of the surgical instrument (collectively referred to as “second planned trajectory information”), or the like, or any combination thereof.

As used herein, the planned entry point may refer to a coordinate of a point on a surface of the subject 470 where the surgical instrument enters the subject 470 in the three-dimensional space (e.g., the world coordinate system as illustrated in FIG. 1). The planned final position may refer to a coordinate of a point inside the subject 470 where the surgical instrument stops entering the subject 470 in the three-dimensional space. The planned trajectory may be a path between the planned entry point and the planned final position in the three-dimensional space. The planned entry depth may refer to a distance between the planned entry point and the planned final position or a length of the planned trajectory. The planned entry angle (e.g., angle (in FIG. 4) may refer to an angle between the planned trajectory and the horizontal direction (i.e., the X direction).

In some embodiments, an operator (e.g., a doctor) may determine the at least one path based on his/her experience and the at least one image. In some embodiments, the at least one path may be automatically determined using a machine learning model. For example, the machine learning model may include a convolutional neural network, an adaptive boosting model, a gradient boosting decision tree model, or the like, or any combination thereof. In some embodiments, the at least one path may be determined by, e.g., a user, with the assistance of a virtual reality device. For instance, a path with respect to a 3D image may be determined by, e.g., a user with the assistance of 3D glasses.

In some embodiments, the length of the surgical instrument may be equal to or greater than the planned entry depth of the surgical instrument. The length of the surgical instrument may be selected based on practical demands, e.g., the aim of the medical procedure, a preference of an operator, a preference of the subject 470, etc.

In some embodiments, the planned trajectory and/or the trajectory information may be determined based on at least one path with respect to the at least one image. As used herein, each of the at least one path may indicate at least a portion of the planned trajectory information of the subject 470. In some embodiments, the at least one image may include at least two 2D images obtained from at least two first views (e.g., a front view, a side view). At least two paths with respect to the at least two 2D images may be determined. A path may be regarded as a representation of the planned trajectory from one of the at least two first views. As used herein, a representation of a trajectory (e.g., a planned trajectory) refers to at least a portion of the trajectory projected from a first view.

Each 2D image of the at least two 2D images may correspond to a two-dimensional space. A path in the image may be a path in the two-dimensional space. As used herein, the two-dimensional space may relate to a first view from which the image is obtained. Taking the world coordinate illustrated in FIG. 1 as an example, the front image may correspond to a two-dimensional space, i.e., the XY coordinate system. The lateral image may correspond to a two-dimensional space, i.e., the XZ coordinate system. Accordingly, a first path with respect to the front image may be a path in the XY coordinate system. A second path with respect to the lateral image may be a path in the XZ coordinate system. The first path may be regarded as a representation of the planned trajectory in the XY coordinate system. The second path may be regarded as a representation of the planned trajectory in the XZ coordinate system.

Therefore, the planned trajectory and/or the first trajectory information may be determined based on the at least two paths. In some embodiments, the planned trajectory and/or the first trajectory information may be determined using a coordinate transformation algorithm, e.g., a grid algorithm, a multi-parameter algorithm, a multi-regression algorithm. For example, the grid algorithm may include a Bursa model, a Morokinsky model, a Weiss model, a Fan's model, etc.

In some embodiments, the at least one image may include at least one 3D image. At least one path with respect to the at least one image may be determined. For each image of the at least one 3D image, since the image is a 3D image, a path with respect to the image may be a path in the three-dimensional space (e.g., the world coordinate system). Each of the at least one path may be the planned trajectory, and the first planned trajectory information may be determined similarly to the process for determining the first planned trajectory information based on the at least two 2D images described above.

In some embodiments, the at least one image may include a 2D image. A path with respect to the 2D image may be obtained. The path may be regarded as a representation of the planned trajectory from a second view. As used herein, the second view may be the same or different from one of the at least one first views. In some embodiments, the second planned trajectory information may be determined based on the path. As used herein, the second planned trajectory information may refer to a probable range of the planned trajectory.

During the medical procedure, the planned trajectory information and/or the at least one path may be used to guide for placing the surgical instrument in the subject 470. The light emitting component 410 may emit a light beam (e.g., a light beam) towards the subject 470. The light beam may be used to indicate the orientation of an actual trajectory to assist in placing the surgical instrument in the subject 470. To ensure the accuracy of the medical procedure, the actual trajectory may need to match the at least one path and/or the planned trajectory information. Accordingly, when the light beam matches the at least one path and/or the planned trajectory information, the medical procedure may be implemented. In some embodiments, a configuration of the light emitting component 410 may be adjusted such that the light beam matches the at least one path (or a portion thereof) and/or the planned trajectory information (or a portion thereof). As used herein, a configuration of the light emitting component 410 may refer to a position (e.g., a position in the three-dimensional space) and an emitting angle of the light emitting component 410.

When the at least one image includes the at least two 2D images, the actual trajectory may be the same as the planned trajectory, or a portion thereof. An intersection of the light beam and the subject 470 may be the planned entry point of the surgical instrument described above. An angle of the light beam in the 3D space may be the planned entry angle of the surgical instrument described above. In some embodiments, the angle of the light beam may be assessed with respect to a surface of the subject 470 in the 3D space. The light beam may indicate the orientation of the actual trajectory of the surgical instrument. For illustration purposes, if a light beam 480 as shown in FIG. 4 matches the at least one path and/or the planned trajectory information, the angle may be a.

In some embodiments, the medical system 100 may also include an image processing component 460. The image processing component 460 may be configured to display at least one representation of the light beam in the at least two 2D images in real-time. In some embodiments, the at least two 2D images may be obtained from different views. For example, the at least two 2D images may include a front image obtained from a front view, a lateral image obtained from a side view, etc. In some embodiments, the configuration of the frame (e.g., the first arm 421 and the second arm 422) and the configuration of the light emitting component 410 may be determined in a first coordinate system with respect to the frame, e.g., using a camera. For example, the configuration of the frame may include a position of the frame, an emitting angle of the frame, an orientation of the frame, etc. For example, a first initial configuration of the frame and a second initial configuration of the light emitting component 410 may be determined with respect to a reference configuration (e.g., an original configuration (0, 0, 0) of the first coordinate system. A change of the configuration of the frame and a change of the configuration of the light emitting component with respect to the original configuration may be determined, e.g., via a sensor, while rotating and/or moving the frame and/or the light emitting component. Since a relationship between the first coordinate system and a second coordinate system with respect to the at least two 2D images may be known, the configuration of the light emitting component 410 with respect to the second coordinate system may be determined. The configuration of the light emitting component 410 may be displayed in the at least two 2D images. The at least one representation of the light beam may also be determined and displayed in the at least two 2D images. In some embodiments, a trajectory of the light beam may depend on the configuration of the light emitting component 410. The image processing component 460 may determine the trajectory of the light beam based on the configuration of the light emitting component 410. The image processing component 460 may further determine the at least two representations of the light beam in the at least two 2D images. When the configuration of the light emitting component 410 is adjusted, the at least two representations of the light beam in the at least one image may change. When one of the at least two representations of the light beam in the at least two 2D images overlaps a corresponding path, the light beam may match the at least two paths. At this time, the light beam may indicate the orientation of the actual trajectory of the surgical instrument.

When the at least one image includes the at least one 3D image, the actual trajectory may be the same as the planned trajectory, i.e., the path. An intersection of the light beam and the subject 470 may be the planned entry point of the surgical instrument described above. An angle of the light beam in the 3D space may be the planned entry angle of the surgical instrument described above. In some embodiments, the angle of the light beam may be assessed with respect to a surface of the subject 470 in the 3D space. The light beam may indicate the actual trajectory of the surgical instrument. The image processing component 460 may display the light beam in the at least one 3D image in real-time. When the configuration of the light emitting component 410 is adjusted, the light beam in the at least one 3D image may change. When one of the light beams in one of the at least one 3D image overlaps the path, i.e., the planned trajectory, the light beam may match the path. At this time, the light beam may indicate the orientation of the actual trajectory of the surgical instrument.

In some embodiments, the image processing component 460 may directly determine the configuration of the light emitting component 410 for placing the surgical instrument based on the first planned trajectory information. Accordingly, the light emitting component 410 may be directly adjusted to the configuration.

It should be noted when the at least one image includes the at least two 2D images or the at least one 3D image, it may be unnecessary to display the light beam and/or the representation of the light beam in the at least one image in real-time during the medical procedure. However, when the light beam and/or the representation of the light beam is displayed in the at least one image in real-time, an operator may visually observe the at least one image to confirm whether the surgical instrument is placed accurately.

When the at least one image includes the 2D image, a representation of the light beam may be displayed in the at least one image. When the representation of the light beam on the 2D image overlaps the path, the light beam may match the path. At this time, the light beam may indicate the orientation of the actual trajectory of the surgical instrument.

In some embodiments, although the planned final position of the surgical instrument is unknown, a position of the subject 470 corresponding to the final position may be determined. The position may be placed at the center (i.e., (x₀, y₀, z₀)) of the three-dimensional space. The center of the 2D image may also be at the position. For example, the image may be the front image, and the front image may be in the xz plane. The center of the front image may be (x₀, z₀). Assuming that the path is from (x₁, z₁) to (x₂, z₂), a path in the three-dimensional space corresponding to the path may be from (x₁, y₀, z₁) to (x₂, y₀, z₂). In order to match the path, the light emitting component 410 may be adjusted in a plane (e.g., an xz plane) vertical to the y-direction and with a value of y₀ in the y-direction. Accordingly, the light emitting component 410 may be adjusted to face the center of the first arm 421 and rotate along the first arm 421 to match the path. In some embodiments, the light emitting component 410 may be unnecessary to face (x₀, y₀, z₀). The position may be placed at a point along the y-direction. The configuration of the light emitting component 410 may also be adjusted based on the process described above.

For illustration purposes, the light emitting component 410 may include a crisscross laser generator, a T laser generator, a dot laser generator, etc. A cross section of the light beam may have a shape of T, a shape of a crisscross, a shape of a dot, etc. A type of the light emitting component 410 may be non-limiting and selected based on practical demands.

The medical system 100 may also include a configuration adjusting component. The configuration adjusting component may be configured to adjust the configuration of the light emitting component 410. In some embodiments, the configuration adjusting component may include a holder 430. The holder 430 may be mounted on the image obtaining assembly 420. For illustration purposes, the holder 430 may be mounted on the first arm 421. The holder 430 may be freely rotated around a position (e.g., an end) of the first arm 421. In some embodiments, the holder 430 may be mounted on the second arm 422. The holder 430 may be freely rotated around on an axis (e.g., a center axis in a horizontal direction) of the second arm 422. For illustration purposes, the holder 430 may have a shape of an arc. A length of the arc may include ¼ of a circle, ½ of a circle. The shape and/or the length of the holder 430 may be non-limiting and selected based on practical demands.

In some embodiments, the light emitting component 410 may be slidably mounted on the holder 430. For illustration purposes, the light emitting component 410 may be mounted on an end of the holder 430. In some embodiments, the configuration of the light emitting component 410 may change by moving the holder 430 and/or sliding and/or rotating the light emitting component 410 along the holder 430. In some embodiments, the medical system 100 may also include a sensor (not shown in FIG. 4). The sensor may be mounted on the holder 430. For illustration purposes, the sensor may be mounted on the light emitting component 410. The sensor may obtain information regarding the configuration of the light emitting component 410.

In some embodiments, the configuration adjusting component may include an automation component (not shown in FIG. 4). The automation component may be mounted on the image obtaining assembly 420 or a component external to the image obtaining assembly. The automation component may be configured to automatically adjust the configuration of the light emitting component 410 (or the light beam). For example, the automation component may include a robotic arm, a robot, etc. In some embodiments, the light emitting component 410 may be mounted on the automation component. The configuration of the light emitting component 410 may be adjusted more freely using the automation component compared to using the holder 430. For example, the automation component may facilitate the light emitting component 410 to be adjusted to any one of more configuration choices than the holder 430.

In some embodiments, the surgical instrument may be placed manually according to the planned trajectory information and/or the at least one path. In some embodiments, the surgical instrument may be mounted inside the surgical instrument placing component 440. For illustration purposes, the surgical instrument placing component 440 may have a shape of a tube. A center axis of the surgical instrument may overlap a center axis of the surgical instrument placing component 440. The surgical instrument placing component 440 may be configured to place the surgical instrument in the subject 470 based at least in part on the light beam. As described above, the light beam may coincide with the at least one path (or the planned trajectory information). The surgical instrument may need to be placed to coincide with at least a portion of the light beam. To ensure the accuracy of the medical procedure, the surgical instrument may be placed to implement the aim of the medical procedure. For illustration purposes, if the planned trajectory information includes a planned entry angle, and the surgical instrument placing component 440 as shown in FIG. 4 coincides with at least a portion of the light beam, β may equal the planned entry angle.

In some embodiments, a configuration of the surgical instrument placing component 440 may be adjusted such that the surgical instrument coincides with the at least a portion of the light beam. As used herein, the configuration of the surgical instrument may refer to a position (e.g., a position in the three-dimensional space) and an angle of the surgical instrument placing component 440. In some embodiments, when the surgical instrument coincides with the at least a portion of the light beam, a first end of the surgical instrument may be placed on the intersection of the light beam and the subject 470. The second end of the surgical instrument may be placed along the at least a portion of the light beam. In some embodiments, an end of the surgical instrument placing component 440 may include a positioning component. As used herein, the end of the surgical instrument placing component 440 may refer to an end of the surgical instrument placing component 440 relatively far from the first end of the surgical instrument. A cross-section of the positioning component may have the same shape and size as the shape and size of the cross-section of the light beam. When the light beam overlaps the cross-section of the positioning component, the second end of the surgical instrument may be placed along the at least a portion of the light beam.

During the medical procedure, if the planned trajectory information includes the planned entry depth of the surgical instrument, the surgical instrument may be placed in the subject 470 according to the planned entry depth. In some embodiments, the surgical instrument may have spaced markings along its length. The actual depth may be adjusted to be the same as the planned entry depth by reading the spaced markings, and the actual depth of the surgical instrument may be the same as or close to the planned entry depth. The deviation of the actual depth of the surgical instrument from the planned entry depth may be negligible or within a medically tolerable range.

In some embodiments, the image obtaining assembly 420 may obtain at least one second image or a video from a selected view. From the selected view, an angle of an emitting direction of the X-ray source 451 and the light beam may be at an angle (e.g., 90 degrees). An actual depth of the surgical instrument may be determined based on the at least one second image or video. For instance, the actual depth may be a length of the surgical instrument represented in the at least one second image or video if the angle is 90 degrees; and the actual depth may be adjusted to be the same as the planned entry depth based on the at least one second image or video. If the angle is not 90 degrees, the actual depth of the surgical instrument may be determined based on the angle and the length of the surgical instrument represented in the at least one second image or video.

In some embodiments, the image obtaining assembly 420 may obtain at least one third image from a third view. As used herein, the third view may be one of the at least two first views. A length of the surgical instrument in the at least one third image may be the same as a length of a path in an image with respect to the first view. The actual depth may be adjusted to be the same as the planned entry depth based on the at least one third image.

During the medical procedure, if the planned trajectory information only includes the planned range of the entry depth of the surgical instrument, the surgical instrument may be placed in the subject 470 according to the path. In some embodiments, the image obtaining assembly 420 may obtain at least one fourth image from the second view. A length of the surgical instrument in the fourth image may be the same as a length of the path.

In some embodiments, although the planned final position of the surgical instrument is unknown, a position of the subject 470 corresponding to the final position may be determined. At least one fifth image from a fifth view may be obtained. Whether the surgical instrument is placed at the position of the subject 470 may be determined based on the at least one fifth image. In some embodiments, the fifth view may be the same as or different from the second view. For example, the fifth view may include the second view, a view from which an emitting direction of the X-ray source 451 and the light beam are at an angle (e.g., an angle of 90 degrees), etc.

In some embodiments, during the medical procedure, at least one sixth image may be obtained to examine whether the medical procedure is implemented according to the planned trajectory information and/or at least one the path. In response to a determination that the medical procedure deviates from the planned trajectory information and/or at least one the path, the medical procedure may be adjusted immediately. In response to a determination that the medical procedure is implemented according to the planned trajectory information and/or at least one the path, the medical procedure may proceed further. In some embodiments, the at least one sixth image may include the at least one second image, the at least one third image the at least one fourth image, the at least one fifth image, or images obtained from other views, etc. In some embodiments, each of the at least one sixth image may represent an anatomical structure of at least a portion (e.g., an organ, tissue) of the subject and the structure of the surgical instrument. By registering the at least one sixth image and the image(s) used to determine the planned trajectory information and/or the at least one the path, at least one actual path of the surgical instrument with respect to the image(s) may be determined. If the at least one actual path coincides with the at least one path in the image(s), the medical procedure may proceed further. In some embodiments, at least one image including the surgical instrument may be obtained by a camera. The at least one image may be used to determine an actual depth of the surgical instrument entered into the subject. For example, the actual depth may be a difference between a length of the surgical instrument and a length of a part of the surgical instrument outside the subject. If the actual length of the surgical instrument is smaller than the planned entry depth, the medical procedure may proceed further.

In some embodiments, an operator may hold the surgical instrument placing component 440 and adjust the configuration of the surgical instrument placing component. In some embodiments, a second automation component may automatically adjust the configuration of the surgical instrument placing component. The second automation component may be the same as or different from the automation component.

In some embodiments, the processing device 140 may obtain at least one seventh image by the image obtaining assembly 420 to confirm whether the medical procedure has been completed as the planned trajectory information and/or the at least one path.

It should be noted that in some embodiments, the subject 470 may be scanned to obtain the at least one image before the initiation of the medical procedure (or referred to as a pre-procedure image for brevity). During the medical procedure, the surgical instrument may be guided by the light beam and the at least one pre-procedure image, thereby obviating the need to scan the subject 470. The time for scanning may be reduced, radiation to the subject 470 (e.g., a patient) may be avoided or reduced, and the utility efficiency of the medical procedure may be improved.

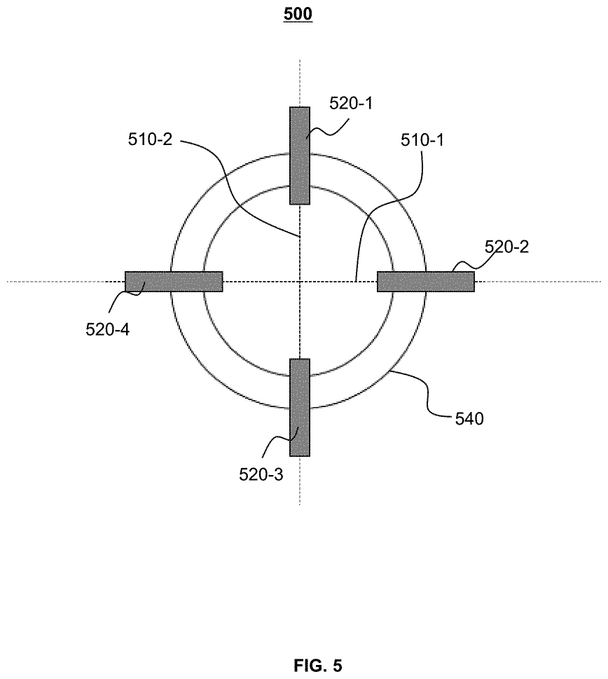

FIG. 5 is a top view illustrating an exemplary end 500 of the surgical instrument placing component 440 according to some embodiments of the present disclosure. The end 500 may be an example of the end of the surgical instrument placing component 440.

As illustrated in FIG. 5, the cross-section of the end 500 may have a shape of a crisscross represented by dash line 510-1 and dash line 510-2. Cross-sections of a component 520-1, a component 520-2, a component 520- and a component 520-4 of the surgical instrument placing component 440 may have the same shape of a slot. A cross-section of a component 540 of the surgical instrument placing component 440 may have a shape of a circle. The component 520-1, the component 520-2, the component 520-3, and the component 520-4 may be situated in four positions of the component 540. A center angle between each two positions may be 90 degrees. Therefore, the shape of the crisscross may form for the end 500.

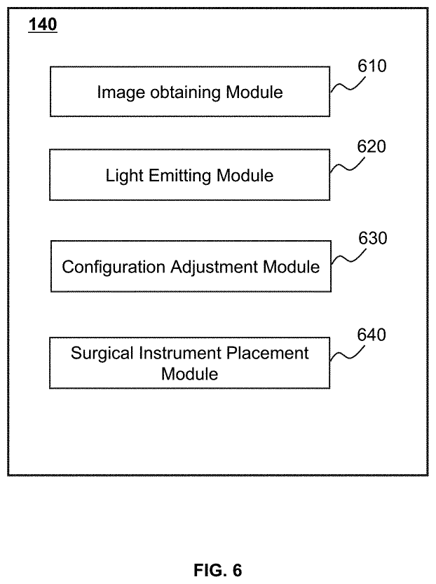

FIG. 6 is a block diagram illustrating an exemplary processing device 140 according to some embodiments of the present disclosure. The processing device 140 may be implemented on the computing device 200 (e.g., the processor 210) as illustrated in FIG. 2 or the CPU 340 as illustrated in FIG. 3. The processing device 140 may include an image obtaining module 610, a light emitting module 620, a configuration adjustment module 630, and a surgical instrument placement module 640.

The modules in the processing device 140 may be connected to or communicate with each other via a wired connection or a wireless connection. The wired connection may include a metal cable, an optical cable, a hybrid cable, or the like, or any combination thereof. The wireless connection may include a Local Area Network (LAN), a Wide Area Network (WAN), a Bluetooth, a ZigBee, a Near Field Communication (NFC), or the like, or any combination thereof.

The image obtaining module 610 may be configured to may obtain at least one image of a subject (e.g., the subject 470 as illustrated in FIG. 4) by an image obtaining assembly (e.g., the image obtaining assembly 110 as illustrated in FIG. 1, the image obtaining assembly 420 as illustrated in FIG. 4). As used herein, each of the at least one image may represent an anatomical structure of at least a portion (e.g., an organ, tissue) of the subject. In some embodiments, the at least one image may include a two-dimensional (2D) image, a three-dimensional (3D) image, etc. In some embodiments, the at least one image may include a PET image, a SPECT image, a CT image (e.g., a CBCT image), an MRI image, a DR (digital radiography) image, an image, a PET-MRI image, or a SPECT-MRI image.

In some embodiments, the at least one image may correspond to at least one view. For example, the at least one image may include a front image obtained from a front view and a lateral image obtained from a side view. As used herein, the image obtaining assembly may obtain the front image when the subject lies on the back, a radiation source (e.g., the X-ray source as illustrated in FIG. 1, the X-ray source as illustrated in FIG. 4) of the image obtaining assembly is along the Z direction as illustrated in FIG. 1. The image obtaining assembly may obtain the lateral image when the subject lies on the side, the radiation source of the image obtaining assembly is along the X direction or the Y direction as illustrated in FIG. 1.

The image obtaining module 610 may determine a treatment plan for a medical procedure for placing a surgical instrument in the subject. For illustration purposes, the treatment plan may include a planned trajectory for placing the surgical instrument in the subject, planned trajectory information for placing the surgical instrument in the subject, a length of the surgical instrument, a configuration of the light emitting component, a configuration of a light beam emitted by the light emitting component 410, etc. In some embodiments, the configuration of the light beam may coincide with the configuration of the light emitting component 410. For example, the configuration of the light beam may include an orientation of the light beam, an emitting angle of the light beam, an emitting direction of the light beam, etc.

As used herein, the planned trajectory and/or the planned trajectory information may indicate how the surgical instrument needs to be placed in the subject during the medical procedure. For example, the planned trajectory information may include a planned entry point (e.g., a point in a three-dimensional space) of the surgical instrument, a planned entry angle of the surgical instrument, a planned entry depth of the surgical instrument, a planned final position (e.g., a position in a three-dimensional space) of the surgical instrument (collectively referred to as “first planned trajectory information”), a planned range of an entry point of the surgical instrument, a planned range of an entry angle of the surgical instrument, a planned range of an entry depth of the surgical instrument, a planned range of a final position of the surgical instrument (collectively referred to as “second planned trajectory information”), or the like, or any combination thereof.

As used herein, the planned entry point may refer to a coordinate of a point on a surface of the subject where the surgical instrument enters the subject in a three-dimensional space (e.g., the world coordinate system as illustrated in FIG. 1). The planned final position may refer to a coordinate of a point inside the subject where the surgical instrument stops entering the subject in a three-dimensional space. The planned trajectory may be a path between the planned entry point and the planned final position in the three-dimensional space. The planned entry depth may refer to a distance between the planned entry point and the planned final position or a length of the planned trajectory. The planned entry angle (e.g., angle (in FIG. 4) may refer to an angle between the planned trajectory and the horizontal direction (i.e., the X direction).

In some embodiments, the length of the surgical instrument may be equal to or greater than the planned entry depth of the surgical instrument. The image obtaining module 610 may select the length of the surgical instrument based on practical demands, e.g., the aim of the medical procedure, a preference of an operator, a preference of the subject, etc.

In some embodiments, the image obtaining module 610 may determine the planned trajectory and/or the trajectory information based on at least one path with respect to the at least one image. As used herein, each of the at least one path may indicate at least a portion of the planned trajectory information of the subject.

The light emitting module 620 may be configured to cause a light emitting component (e.g., a light emitting component as illustrated in FIG. 4) to emit a light beam towards the subject. The light beam may be used to indicate the orientation of an actual trajectory of the surgical instrument to assist in placing the surgical instrument in the subject. To ensure the accuracy of the medical procedure, the actual trajectory may need to match the at least one path and/or the planned trajectory information.

For illustration purposes, the light emitting component may include a crisscross laser generator, a T laser generator, a dot laser generator, etc. A cross section of the light beam may have a shape of T, a shape of a crisscross, a shape of a dot, etc. A type of the light emitting component 410 may be non-limiting and selected based on practical demands. The light emitting component may be similar to or the same as the light emitting component 410 as illustrated in FIG. 4, and not repeated here.

The configuration adjustment module 630 may be configured to adjust a configuration of the light emitting component such that the light beam matches the at least one path in the at least one image. As described above, the light beam may be used to indicate the orientation of an actual trajectory of the surgical instrument to assist in placing the surgical instrument. The actual trajectory may need to match the at least one path and/or the planned trajectory information. In some embodiments, the configuration adjustment module 630 may adjust the configuration of the light emitting component such that the light beam matches the orientation of at least one path and/or the planned trajectory information). As used herein, the configuration of the light emitting component may refer to a position (e.g., a position in the three-dimensional) and an emitting angle of the light emitting component.

When the at least one image includes the at least two 2D images, the actual trajectory may be the same as the planned trajectory or a portion thereof. An intersection of the light beam and the subject may be the planned entry point of the surgical instrument described above. An angle of the light beam in the 3D space may be the planned entry angle of the surgical instrument described above. In some embodiments, the angle of the light beam may be assessed with respect to a surface of the subject 470 in the 3D space. The light beam may indicate the orientation of the actual trajectory of the surgical instrument. For illustration purposes, if light beam 480 as shown in FIG. 4 matches the at least one path and/or the planned trajectory information, the angle may be a.

The configuration adjustment module 630 may display at least one representation of the light beam in the at least two 2D images in real-time. In some embodiments, a trajectory of the light beam may depend on the configuration of the light emitting component. The configuration adjustment module 630 may determine the trajectory of the light beam based on the configuration of the light emitting component 410 by an image processing component (e.g., the image processing component as illustrated in FIG. 4). The image processing component may further determine the at least two representations of the light beam in the at least two 2D images. When one of the at least two representations of the light beam in the at least two 2D images overlaps a corresponding path, the light beam may match the at least two paths. At this time, the light beam may indicate the actual trajectory of the surgical instrument.

When the at least one image includes the at least one 3D image, the actual trajectory may be the same as the planned trajectory, i.e., the path. An intersection of the light beam and the subject may be the planned entry point of the surgical instrument described above. An angle of the light beam in the 3D space may be the planned entry angle of the surgical instrument described above. In some embodiments, the angle of the light beam may be assessed with respect to a surface of the subject 470 in the 3D space. The light beam may indicate the actual trajectory of the surgical instrument. The image processing component may display the light beam in the at least one 3D image in real-time. When the configuration of the light emitting component is adjusted, the light beam in the at least one 3D image may change. When one of the light beams in one of the at least one 3D image overlaps the path, i.e., the planned trajectory, the light beam may match the path. At this time, it may indicate that the orientation of the surgical instrument is in conformity with the orientation of the planned trajectory.

In some embodiments, the image processing component may directly determine the configuration of the light emitting component 410 for placing the surgical instrument based on the first planned trajectory information. Accordingly, the configuration adjustment module 630 may directly adjust the light emitting component 410 to the configuration.

When the at least one image includes the 2D image, the configuration adjustment module 630 may display (the representation of) the light beam in the at least one image. When a representation of the light beam on the 2D image overlaps the path, the light beam may match the path. At this time, the light beam may indicate the actual trajectory of the surgical instrument.

The surgical instrument placement module 640 may be configured to place the surgical instrument in the subject based at least in part on the light beam. As described above, the light beam may coincide with the at least one path (or the planned trajectory information). To ensure the accuracy of the medical procedure, the surgical instrument placement module 640 may place the surgical instrument to coincide with at least a portion of the light beam. For illustration purposes, if the planned trajectory information includes a planned entry angle, and the surgical instrument placing component 440 as shown in FIG. 4 coincides with at least a portion of the light beam, β may equal the planned entry angle.

In some embodiments, the surgical instrument placement module 640 may adjust a configuration of the surgical instrument placing component 440 such that the surgical instrument coincides with the at least a portion of the light beam. As used herein, the configuration of the surgical instrument may refer to a position (e.g., a position in the three-dimensional space) and an angle of the surgical instrument placing component 440. In some embodiments, when the surgical instrument coincides with the at least a portion of the light beam, the surgical instrument placement module 640 may place a first end of the surgical instrument on the intersection of the light beam and the subject. The surgical instrument placement module 640 may place a second end of the surgical instrument along the at least a portion of the light beam.

During the medical procedure, if the planned trajectory information includes the planned entry depth of the surgical instrument, the surgical instrument placement module 640 may place the surgical instrument in the subject according to the planned entry depth. In some embodiments, the surgical instrument may have spaced markings along its length. The surgical instrument placement module 640 may adjust the actual depth to be the same as the planned entry depth by reading the spaced markings, and the actual depth of the surgical instrument may be the same as or close to the planned entry depth. The deviation of the actual depth of the surgical instrument from the planned entry depth may be negligible or within a medically tolerable range.

In some embodiments, the surgical instrument placement module 640 may obtain at least one second image or a video from a selected view by the image obtaining assembly. From the selected view, an angle of an emitting direction of the radiation source and the light beam may be at an angle (e.g., 90 degrees). The surgical instrument placement module 640 may determine the actual depth of the surgical instrument based on the at least one second image or video. If the angle is not 90 degrees, the surgical instrument placement module 640 may determine the actual depth of the surgical instrument based on the angle and the length of the surgical instrument represented in the at least one second image or video. The surgical instrument placement module 640 may adjust the actual depth to be the same as the planned entry depth based on the at least one second image.

In some embodiments, the surgical instrument placement module 640 may obtain at least one third image from a third view. As used herein, the third view may be one of the at least two first views by the image obtaining assembly 420. A length of a representation of the surgical instrument in the at least one third image may be the same as a length of a path in an image with respect to the first view. The surgical instrument placement module 640 may adjust the actual depth to be the same as the planned entry depth based on the at least one third image.

During the medical procedure, if the planned trajectory information only includes the planned range of the entry depth of the surgical instrument, the surgical instrument placement module 640 may place the surgical instrument in the subject according to the path. In some embodiments, the surgical instrument placement module 640 may obtain at least one fourth image from the second view. A length of the surgical instrument in the fourth image may be the same as a length of the path.

In some embodiments, although the planned final position of the surgical instrument is unknown, the surgical instrument placement module 640 may determine a position of the subject corresponding to the final position may be determined. The surgical instrument placement module 640 may obtain at least one fifth image from a fifth view. The surgical instrument placement module 640 may determine whether the surgical instrument is placed at the position of the subject based on the at least one fifth image. In some embodiments, the fifth view may be the same as or different from the second view. For example, the fifth view may include the second view, a view from which an emitting direction of the radiation source and the light beam are at an angle (e.g., 90 degrees), etc.

In some embodiments, during the medical procedure, the surgical instrument placement module 640 may obtain at least one sixth image to assure that the medical procedure is implemented according to the planned trajectory information and/or at least one the path. In some embodiments, the at least one sixth image may include the at least one second image, the at least one third image, the at least one fourth image, the at least one fifth image, or images obtained from other views, etc.

In some embodiments, the surgical instrument placement module 640 may obtain at least one seventh image by the image obtaining assembly to confirm that the medical procedure has been completed as the planned trajectory information and/or the at least one path.

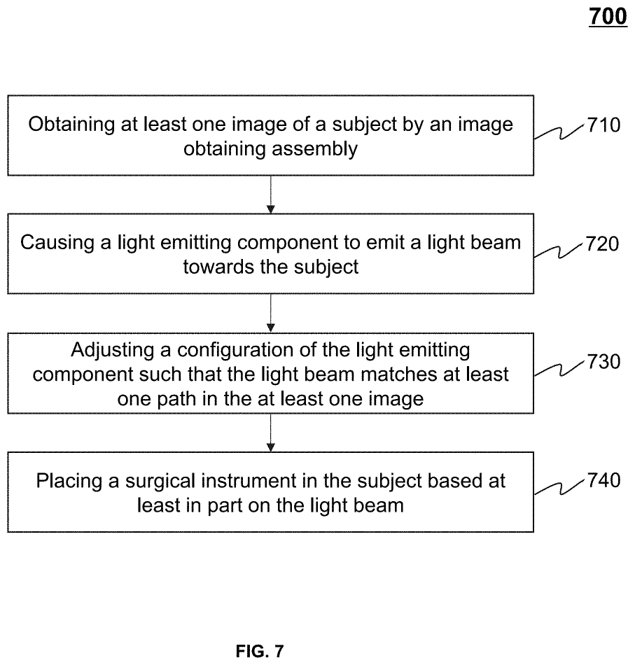

FIG. 7 is a flowchart illustrating an exemplary process 700 for placing a surgical instrument according to some embodiments of the present disclosure. The process 700 may be implemented in the medical system 100 illustrated in FIG. 1. For example, the process 700 may be stored in the storage device 150 and/or the storage 220 in the form of instructions (e.g., an application), and invoked and/or executed by the processing device 140 (e.g., the processor 210 illustrated in FIG. 2, the CPU 340 as illustrated in FIG. 3, or one or more modules in the processing device 140 illustrated in FIG. 6). The operations of the illustrated process presented below are intended to be illustrative. In some embodiments, the process 700 may be accomplished with one or more additional operations not described, and/or without one or more of the operations discussed. Additionally, the order in which the operations of the process 700 as illustrated in FIG. 7 and described below is not intended to be limiting.

In 710, the processing device 140 (e.g., the image obtaining module 610) may obtain at least one image of a subject (e.g., the subject 470 as illustrated in FIG. 4) by an image obtaining assembly (e.g., the image obtaining assembly 110 as illustrated in FIG. 1, the image obtaining assembly 420 as illustrated in FIG. 4). As used herein, each of the at least one image may represent an anatomical structure of at least a portion (e.g., an organ, tissue) of the subject. In some embodiments, the at least one image may include a two-dimensional (2D) image, a three-dimensional (3D) image, etc. In some embodiments, the at least one image may include a PET image, a SPECT image, a CT image (e.g., a CBCT image), an MRI image, a DR (digital radiography) image, an image, a PET-MRI image, or a SPECT-MRI image.

In some embodiments, the at least one image may correspond to at least one view. For example, the at least one image may include a front image obtained from a front view and a lateral image obtained from a side view. As used herein, the image obtaining assembly may obtain the front image when the subject lies on the back, a radiation source (e.g., the X-ray source as illustrated in FIG. 1, the X-ray source as illustrated in FIG. 4) of the image obtaining assembly is along the Z direction as illustrated in FIG. 1. The image obtaining assembly may obtain the lateral image when the subject lies on the side, the radiation source of the image obtaining assembly is along the X direction or the Y direction as illustrated in FIG. 1.

The processing device 140 may determine a treatment plan for a medical procedure for placing a surgical instrument in the subject. For illustration purposes, the treatment plan may include a planned trajectory for placing the surgical instrument in the subject, planned trajectory information for placing the surgical instrument in the subject, a length of the surgical instrument, a configuration of the light emitting component, a configuration of a light beam emitted by the light emitting component, etc. In some embodiments, the configuration of the light beam may coincide with the configuration of the light emitting component. For example, the configuration of the light beam may include an orientation of the light beam, an emitting angle of the light beam, an emitting direction of the light beam, etc.

As used herein, the planned trajectory and/or the planned trajectory information may indicate how the surgical instrument needs to be placed in the subject during the medical procedure. For example, the planned trajectory information may include a planned entry point (e.g., a point in a three-dimensional space) of the surgical instrument, a planned entry angle of the surgical instrument, a planned entry depth of the surgical instrument, a planned final position (e.g., a position in a three-dimensional space) of the surgical instrument (collectively referred to as “first planned trajectory information”), a planned range of an entry point of the surgical instrument, a planned range of an entry angle of the surgical instrument, a planned range of an entry depth of the surgical instrument, a planned range of a final position of the surgical instrument (collectively referred to as “second planned trajectory information”), or the like, or any combination thereof.

As used herein, the planned entry point may refer to a coordinate of a point on a surface of the subject where the surgical instrument enters the subject in a three-dimensional space (e.g., the world coordinate system as illustrated in FIG. 1). The planned final position may refer to a coordinate of a point inside the subject where the surgical instrument stops entering the subject in a three-dimensional space. The planned trajectory may be a path between the planned entry point and the planned final position in the three-dimensional space. The planned entry depth may refer to a distance between the planned entry point and the planned final position or a length of the planned trajectory. The planned entry angle (e.g., angle (in FIG. 4) may refer to an angle between the planned trajectory and the horizontal direction (i.e., the X direction).

In some embodiments, the length of the surgical instrument may be equal to or greater than the planned entry depth of the surgical instrument. The processing device 140 may select the length of the surgical instrument based on practical demands, e.g., the aim of the medical procedure, a preference of an operator, a preference of the subject, etc.

In some embodiments, the processing device 140 may determine the planned trajectory and/or the trajectory information based on at least one path with respect to the at least one image. As used herein, each of the at least one path may indicate at least a portion of the planned trajectory information of the subject. In some embodiments, the at least one image may include at least two 2D images obtained from at least two first views (e.g., a front view, a side view). The processing device 140 may determine at least two paths with respect to the at least two 2D images. The processing device 140 may regard a path as a representation of the planned trajectory from one of the at least two first views. As used herein, a representation of a trajectory (e.g., a planned trajectory) refers to at least a portion of the trajectory projected from a first view.

Each 2D image of the at least two 2D images may correspond to a two-dimensional space. A path in the image may be a path in the two-dimensional space. As used herein, the two dimension space may relate to a first view from which the image is obtained. Taking the world coordinate illustrated in FIG. 1 as an example, the front image may correspond to a two-dimensional space, i.e., the XY coordinate system. The lateral image may correspond to a two-dimensional space, i.e., the XZ coordinate system. Accordingly, a first path with respect to the front image may be a path in the XY coordinate system. A second path with respect to the lateral image may be a path in the XZ coordinate system. The processing device 140 may regard the first path as a representation of the planned trajectory in the XY coordinate system. The processing device 140 may regard the second path as a representation of the planned trajectory in the XZ coordinate system.

Therefore, the processing device 140 may determine the planned trajectory and/or the first trajectory information based on the at least two paths. In some embodiments, the processing device may determine the planned trajectory and the first trajectory information using a coordinate transformation algorithm, e.g., a grid algorithm, a multi-parameter algorithm, a multi-regression algorithm. For example, the grid algorithm may include a Bursa model, a Morokinsky model, a Weiss model, a Fan's model, etc. In some embodiments, the at least one path may be determined by, e.g., a user, with the assistance of a virtual reality device. For instance, a path with respect to a 3D image may be determined by, e.g., a user with the assistance of 3D glasses.

In some embodiments, the at least one image may include at least one 3D image. The processing device 140 may determine at least one path with respect to the at least one image. For each image of the at least one 3D image, since the image is a 3D image, a path with respect to the image may be a path in the three-dimensional space (e.g., the world coordinate system). The processing device 140 may regard each of the at least one path as the planned trajectory. The processing device 140 may determine the first planned trajectory information similarly to the process for determining the first planned trajectory information based on the at least two 2D images described above.

In some embodiments, the at least one image may include a 2D image. The processing device 140 may obtain a path with respect to the 2D image. The processing device 140 may regard the path as a representation of the planned trajectory from a second view. As used herein, the second view may be the same or different from one of the at least one first views. The processing device 140 may be unlikely to determine the planned trajectory and the first planned trajectory information based on the path. In some embodiments, the processing device 140 may determine the second planned trajectory information based on the path. As used herein, the second planned trajectory information may refer to a probable range of the planned trajectory. In some embodiments, the planned trajectory information may be determined manually by a clinician, or automatically by a computing unit, for example, a machine learning model.

In 720, the processing device 140 (e.g., the light emitting module 620) may cause a light emitting component (e.g., a light emitting component as illustrated in FIG. 4) to emit a light beam (e.g., a light beam) towards the subject. The light beam may be used to indicate the orientation of an actual trajectory of the surgical instrument to assist in placing the surgical instrument in the subject. To ensure the accuracy of the medical procedure, the actual trajectory may need to match the at least one path and/or the planned trajectory information.

For illustration purposes, the light emitting component may include a crisscross laser generator, a T laser generator, a dot laser generator, etc. A cross section of the light beam may have a shape of T, a shape of a crisscross, a shape of a dot, etc. A type of the light emitting component 410 may be non-limiting and selected based on practical demands. The light emitting component may be similar to or the same as the light emitting component 410 as illustrated in FIG. 4, and not repeated here.

In 730, the processing device 140 (e.g., the configuration adjustment module 630) may adjust a configuration of the light emitting component such that the light beam matches the at least one path in the at least one image. As described above, the light beam may be used to indicate an actual trajectory of the surgical instrument to assist in placing the surgical instrument. The actual trajectory may need to match the at least one path and/or the planned trajectory information. In some embodiments, the processing device 140 may adjust the configuration of the light emitting component such that the light beam matches the at least one path and/or the planned trajectory information). As used herein, the configuration of the light emitting component may refer to a position (e.g., a position in the three-dimensional) and an emitting angle of the light emitting component.

When the at least one image includes the at least two 2D images, the actual trajectory may be the same as the planned trajectory, or a portion thereof. An intersection of the light beam and the subject may be the planned entry point of the surgical instrument described above. An angle of the light beam in the 3D space may be the planned entry angle of the surgical instrument described above. In some embodiments, the angle of the light beam may be assessed with respect to a surface of the subject 470 in the 3D space. The light beam may indicate the actual trajectory of the surgical instrument.

In some embodiments, the medical system 100 may also include an image processing component (e.g., the image processing component 460 as illustrated in FIG. 4). The processing device 140 may display at least one representation of the light beam in the at least two 2D images in real-time by the image processing component. In some embodiments, the processing device 140 may obtain the at least two 2D images of different views. For example, the at least two 2D images may include a front image obtained from a front view, a lateral image obtained from a side view, etc. In some embodiments, the processing device 140 may determine the configuration of the frame (e.g., the first arm 421 and the second arm 422) and the configuration of the light emitting component 410 in a first coordinate system with respect to the frame, e.g., using a camera. For example, the configuration of the frame may include a position of the frame, an emitting angle of the frame, an orientation of the frame, etc. For example, the processing device 140 may determine a first initial configuration of the frame and a second initial configuration of the light emitting component 410 with respect to a reference configuration (e.g., an original configuration (0, 0, 0) of the first coordinate system. The processing device 140 may determine, e.g., via a sensor, a change of the configuration of the frame and a change of the configuration of the light emitting component with respect to the original configuration while rotating and/or moving the frame and/or the light emitting component. Since a relationship between the first coordinate system and a second coordinate system with respect to the at least two 2D images may be known, the processing device 140 may determine the configuration of the light emitting component 410 with respect to the second coordinate system. The processing device 312 may display the configuration of the light emitting component 410 in the at least two 2D images. The processing device may also determine and display at least one representation of the light beam in the at least two 2D images. In some embodiments, a trajectory of the light beam may depend on the configuration of the light emitting component. The image processing component may determine the trajectory of the light beam based on the configuration of the light emitting component. The image processing component may further determine the at least two representations of the light beam in the at least two 2D images. When the configuration of the light emitting component is adjusted, the at least two representations of the light beam from the at least one image may change. When one of the at least two representations of the light beam in the at least two 2D images overlaps a corresponding path, the light beam may match the at least two paths. At this time, the light beam may indicate the orientation of the actual trajectory of the surgical instrument. For illustration purposes, if light beam 480 as shown in FIG. 4 matches the at least one path and/or the planned trajectory information, the angle may be a.

When the at least one image includes the at least one 3D image, the actual trajectory may be the same as the planned trajectory, i.e., the path. An intersection of the light beam and the subject may be the planned entry point of the surgical instrument described above. An angle of the light beam may be the planned entry angle of the surgical instrument described above. In some embodiments, the angle of the light beam may be assessed with respect to a surface of the subject 470 in the 3D space. The light beam may indicate the actual trajectory of the surgical instrument. The image processing component may display the light beam in the at least one 3D image in real-time. When the configuration of the light emitting component is adjusted, the light beam in the at least one 3D image may change. When one of the light beams in one of the at least one 3D image overlaps the path, i.e., the planned trajectory, the light beam may match the path. At this time, the light beam may indicate the orientation of the actual trajectory of the surgical instrument.

In some embodiments, the image processing component may directly determine the configuration of the light emitting component 410 for placing the surgical instrument based on the first planned trajectory information. Accordingly, the processing device 140 may directly adjust the light emitting component to the configuration.

It should be noted when the at least one image includes the at least two 2D images or the at least one 3D image, the processing device 140 may be unnecessary to display the light beam and/or the representation of the light beam in the at least one image in real-time during the medical procedure. However, when the light beam and/or the representation of the light beam is displayed in the at least one image in real-time, an operator may visually observe the at least one image to confirm whether the surgical instrument is placed accurately.

When the at least one image includes the 2D image, the processing device 140 may display a representation of the light beam in the at least one image. When the representation of the light beam on the 2D image overlaps the path, the light beam may match the path. At this time, the light beam may indicate the orientation of the actual trajectory of the surgical instrument.

In some embodiments, although the planned final position of the surgical instrument is unknown, the processing device 140 determines a position of the subject corresponding to the final position. The processing device 140 may place the position at the center (i.e., (x₀, y₀, z₀)) of the three-dimensional space. The center of the 2D image may also be at the position. For example, the image may be the front image, and the front image may be in the xz plane. The center of the front image may be (x₀, z₀). Assuming that the path is from (x₁, z₁) to (x₂, z₂), a path in the three-dimensional space corresponding to the path may be from (x₁, y₀, z₁) to (x₂, y₀, z₂). In order to match the path, the processing device 140 may adjust the light emitting component in a plane (e.g., an xz plane) vertical to the y-direction and with a value of y₀ in the y-direction. Accordingly, the processing device 140 may adjust the light emitting component to face the center of the first arm 421 and rotate along the first arm 421 to match the path. In some embodiments, the light emitting component may be unnecessary to face (x₀, y₀, z₀). The processing device 140 may place the position at a point along the y-direction. The processing device 140 may also adjust the configuration of the light emitting component based on the process described above.

In 740, the processing device 140 (e.g., the surgical instrument placement module 610) may place the surgical instrument in the subject based at least in part on the light beam. As described above, the light beam may coincide with the at least one path (or the planned trajectory information). To ensure the accuracy of the medical procedure, the processing device 140 may place the surgical instrument to coincide with at least a portion of the light beam. For illustration purposes, if the planned trajectory information includes a planned entry angle, and the surgical instrument placing component 440 as shown in FIG. 4 coincides with at least a portion of the light beam, β may equal the planned entry angle.

In some embodiments, the processing device 140 may adjust a configuration of the surgical instrument placing component such that the surgical instrument coincides with the at least a portion of the light beam. As used herein, the configuration of the surgical instrument may refer to a position (e.g., a position in the three-dimensional space) and an angle of the surgical instrument placing component 440. In some embodiments, when the surgical instrument coincides with the at least a portion of the light beam, the processing device 140 may place a first end of the surgical instrument on the intersection of the light beam and the subject. The processing device 140 may place a second end of the surgical instrument along the at least a portion of the light beam.

During the medical procedure, if the planned trajectory information includes the planned entry depth of the surgical instrument, the processing device 140 may place the surgical instrument in the subject according to the planned entry depth. In some embodiments, the surgical instrument may have spaced markings along its length. The processing device 140 may adjust the actual depth to be the same as the planned entry depth by reading the spaced markings, and the actual depth of the surgical instrument may be the same as or close to the planned entry depth. The deviation of the actual depth of the surgical instrument from the planned entry depth may be negligible or within a medically tolerable range. In some embodiments, the processing device 140 may obtain at least one second image or a video from a selected view by the image obtaining assembly. From the selected view, an angle of an emitting direction of the radiation source and the light beam may be at an angle (e.g., 90 degrees). The processing device 140 may determine the actual depth of the surgical instrument based on the at least one second image or video. The processing device 140 may adjust the actual depth to be the same as the planned entry depth based on the at least one second image or video. If the angle is not 90 degrees, the processing device 140 may determine the actual depth of the surgical instrument based on the angle and the length of the surgical instrument represented in the at least one second image or video.

In some embodiments, the processing device 140 may obtain at least one third image from a third view. As used herein, the third view may be one of the at least two first views by the image obtaining assembly 420. A length of a representation of the surgical instrument in the at least one third image may be the same as a length of a path in an image with respect to the first view. The processing device 140 may adjust the actual depth to be the same as the planned entry depth based on the at least one third image.

During the medical procedure, if the planned trajectory information only includes the planned range of the entry depth of the surgical instrument, the processing device 140 may place the surgical instrument in the subject according to the path. In some embodiments, the processing device 140 may obtain at least one fourth image from the second view. A length of the surgical instrument in the fourth image may be the same as a length of the path.

In some embodiments, although the planned final position of the surgical instrument is unknown, the processing device 140 may determine a position of the subject corresponding to the final position. The processing device 140 may obtain at least one fifth image from a fifth view. The processing device 140 may determine whether the surgical instrument is placed at the position of the subject based on the at least one fifth image. In some embodiments, the fifth view may be the same as or different from the second view. For example, the fifth view may include the second view, a view from which an emitting direction of the radiation source and the light beam are at an angle (e.g., an angle of 90 degrees), etc.

In some embodiments, during the medical procedure, the processing device 140 may obtain at least one sixth image to assure that the medical procedure is implemented according to the planned trajectory information and/or at least one the path. In some embodiments, the at least one sixth image may include the at least one second image, the at least one third image, the at least one fourth image, the at least one fifth image, or images obtained from other views, etc. In some embodiments, each of the at least one sixth image may represent an anatomical structure of at least a portion (e.g., an organ, tissue) of the subject and the structure of the surgical insturment. By registering the at least one sixth image and the image(s) used to determine the planned trajectory information and/or the at least one the path, the processing device 140 may determine at least one actual path of the surgical instrument with respect to the image(s). If the at least one actual path coincides with the at least one path in the image(s), the medical procedure may proceed further. In some embodiments, the processing device 140 may obtain at least one image including the surgical instrument by a camera. The processing device 140 may user the at least one image to determine an actual depth of the surgical instrument entered into the subject. For example, the actual depth may be a difference between a length of the surgical instrument and a length of a part of the surgical instrument outside the subject. If the actual length of the surgical instrument is smaller than the planned entry depth, the medical procedure may proceed further.

In some embodiments, the processing device 140 may obtain at least one seventh image by the image obtaining assembly to confirm that the medical procedure has been completed as the planned trajectory information and/or the at least one path.

It should be noted that the above description is merely provided for the purposes of illustration, and not intended to limit the scope of the present disclosure. For persons having ordinary skills in the art, multiple variations or modifications may be made under the teachings of the present disclosure. However, those variations and modifications do not depart from the scope of the present disclosure. For example, one or more of the operations in process 700 may be performed by an operator (e.g., a doctor), e.g., operation 720, operation 730, operation 740.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A non-transitory computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities, properties, and so forth, used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the descriptions, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A system for placing a surgical instrument in a subject, comprising:
an image obtaining assembly configured to obtain at least one image relating to the subject;
a light emitting component configured to emit a light beam towards the subject; and
a configuration adjusting component configured to adjust a configuration of the light emitting component such that the light beam matches at least one path in the at least one image, wherein each of the at least one path indicates at least a portion of planned trajectory information for placing the surgical instrument in the subject in at least one of the at least one image.

2. The system of claim 1, wherein the planned trajectory information includes at least one of a planned entry point of the surgical instrument, a planned entry angle of the surgical instrument, a planned entry depth of the surgical instrument, a planned final position of the surgical instrument, a planned range of an entry point of the surgical instrument, a planned range of an entry angle of the surgical instrument, a planned range of an entry depth of the surgical instrument, or a planned range of a final position of the surgical instrument.

3. The system of claim 2, wherein the system further includes an image processing component, and
to adjust a configuration of the light emitting component such that the light beam matches at least one path in the at least one image,
the image processing component is configured to display at least one of the at least one image and representation of the light beam on the at least one of the at least one image; and
the configuration adjusting component is configured to adjust the configuration of the light emitting component such that the representation of the light beam coincides with the at least one path in the at least one of the at least one image.

4. The system of claim 2, wherein to adjust a configuration of the light beam such that the light beam matches at least one path in the at least one image, the configuration adjusting component is further configured to:
adjust the configuration of the light emitting component such that the light beam coincides with at least one of the planned entry point of the surgical instrument, the planned entry angle of the surgical instrument, or the planned final position of the surgical instrument.

5. The system of claim 2, wherein the system further includes a surgical instrument placing component configured to place, based at least in part on the light beam, the surgical instrument in the subject.

6. The system of claim 5, wherein to place, based at least in part on the light beam, the surgical instrument in the subject, the surgical instrument placing component is further configured to:
adjust a configuration of the surgical instrument placing component such that the surgical instrument coincides with at least a portion of the light beam; and
place the surgical instrument in the subject.

7. The system of claim 6, wherein to place the surgical instrument, the surgical instrument placing component is further configured to:
place a first end of the surgical instrument on an intersection of the light beam and the subject; and
place a second end of the surgical instrument along the at least a portion of the light beam.

8. The system of claim 7, wherein the surgical instrument placing component is further configured to:
place the surgical instrument in the subject according to the planned entry depth.

9. The system of claim 1, wherein the image obtaining assembly includes at least one of: an X-ray machine, a computed tomography (CT), a positron emission tomography (PET), a single photon emission computed tomography (SPECT), or a magnetic resonance (MR).

10. (canceled)

11. The system of claim 1, the image obtaining assembly further comprising a frame, wherein the light emitting component is mounted on the frame.

12. The system of claim 1, wherein a cross section of the light beam has a shape of T, a shape of a crisscross, or a shape of a dot.

13. The system of claim 1, wherein the configuration adjusting component includes a holder mounted on the image obtaining assembly, and the light emitting component is mounted on the holder.

14. The system of claim 1, wherein
the configuration adjusting component includes an automation component,
the light emitting component is mounted on the automation component, and
the automation component is configured to automatically adjust the configuration of the light emitting component.

15. The system of claim 14, wherein the automation component is mounted on the image obtaining assembly or a component external to the image obtaining assembly.

16. The system of claim 5, wherein the surgical instrument is mounted inside the surgical instrument placing component.

17. The system of claim 16, wherein
the surgical instrument placing component has a shape of a tube, and
a center axis of the surgical instrument placing component overlaps a center axis of the surgical instrument.

18. The system of claim 5, wherein the surgical instrument placing component includes a second automation component configured to automatically adjust the configuration of the surgical instrument placing component.

19. The system claim 1, wherein the surgical instrument includes at least one of a needle, a nail, a screw, or a drill.

20. A method for placing a surgical instrument in a subject, comprising:
obtaining at least one image of a subject by an image obtaining assembly;
causing a light emitting component to emit a light beam towards the subject; and
adjusting a configuration of the light emitting component such that the light beam matches at least one path in the at least one image, wherein each of the at least one path indicates at least a portion of planned trajectory information for placing the surgical instrument in the subject in at least one of the at least one image.

21-32. (canceled)

33. A non-transitory computer readable medium, comprising:
instructions being executed by at least one processor, causing the at least one processor to implement a method, comprising:
obtaining at least one image of a subject by an image obtaining assembly;
causing a light emitting component to emit a light beam towards the subject; and
adjusting a configuration of the light emitting component such that the light beam matches at least one path in the at least one image, wherein each of the at least one path indicates at least a portion of planned trajectory information for placing the surgical instrument in the subject in at least one of the at least one image.
